(19) United States
(12) Patent Application Publication
(10) Pub. No.: US 2012/0022172 A1
(43) Pub. Date: Jan. 26, 2012

(54) METHOD FOR PROVIDING AN ENERGY CARRIER

(75) Inventor: Peter Grauer, Meggen (CH)

(73) Assignee: Silicon Fire AG, Meggen (CH)

(21) Appl. No.: 13/140,823

(22) PCT Filed: Dec. 18, 2008
(86) PCT No.: PCT/EP2008/067895

§ 371 Date: Sep. 29, 2011

(51) Int. Cl. C07C 27/00

(52) U.S. Cl. 518/702

(57) ABSTRACT

A method for providing storable and transportable energy carriers (103, 104) is described. In one step, a transformation of a silicon-dioxide-containing starting material (101) to silicon (103) occurs in a reduction process (105), wherein the primary energy for this reduction process (105) is provided from a renewable energy source. A portion of the reaction products (102) of the reduction process (105) is then applied in a process (106) for generating methanol, wherein a synthesis gas (110) composed of carbon monoxide and hydrogen is used.

### CROSS-REFERENCES TO RELATED APPLICATIONS

The present application claims the priorities of Patent Cooperation Treaty Application No. PCT/EP2008/067895, filed Dec. 18, 2008, and is incorporated herein by reference in its entirety for all purposes.

### FIELD OF THE INVENTION

The present application relates to methods for providing storable and transportable energy carriers.

### BACKGROUND OF THE INVENTION

Carbon dioxide (often called carbonic acid gas) is a chemical compound composed of carbon and oxygen. Carbonic acid gas is a color- and odorless gas. It is a natural component of the air in a small concentration and is generated in animals during the cell respiration, but also in the combustion of carbon-containing substances under supply of sufficient oxygen. Since the advent of the industrialization, the proportion of CO₂ in the atmosphere has risen significantly. A main cause for this are the CO₂ emissions caused by human beings—the so-called anthropogenic CO₂ emissions. The carbonic acid gas in the atmosphere absorbs a portion of the heat radiation. This property renders carbonic acid gas to be a so-called greenhouse gas and is one of the co-originators of the greenhouse effect.

For these and also for other reasons, research and development is performed at present in different directions, to find a way to reduce the anthropogenic CO₂ emissions. In particular in connection with the generation of energy, which is often carried out by the combustion of fossil energy carriers such as coal or gas, but also in other combustion processes, for example in waste incineration, there is a great demand for CO₂ reduction. By such processes, billions of tons of CO₂ are emitted into the atmosphere per year.

### SUMMARY OF THE INVENTION

Now, it is an object to provide a method that is capable of generating other energy carriers, for example as fuels or combustibles. These energy carriers are preferably without emission of CO₂.

According to the invention, a method is proposed for providing storable and renewable energy carriers. In one step, a transformation of silicon-dioxide-containing starting material to silicon occurs in a reduction process, wherein the primary energy for this reduction process is provided from a renewable energy source. A portion of the reaction products of the reduction process is then utilized in a process for generating methanol, wherein in this process for generating methanol, a synthesis gas composed of carbon monoxide and hydrogen is used.

Further preferable embodiments can be taken from the description, the Figures and the dependent claims.

### BRIEF DESCRIPTION OF THE DRAWING

In the drawings, the different aspects of the invention are shown schematically, wherein:

FIG. 1: shows the basic steps of a first method according to the invention;

FIG. 2: shows the basic steps of a second method according to the invention;

FIG. 3: shows the basic steps of a third method according to the invention;

FIG. 4: shows the basic steps of a fourth method according to the invention;

FIG. 5: shows the basic steps of a fifth method according to the invention;

FIG. 6: shows partial steps of a further method according to the invention; and

FIG. 7: shows partial steps of a further method according to the invention.

### DETAILED DESCRIPTION OF THE PREFERRED EMBODIMENTS

The method according to the invention is based on a novel concept, which provides, as a result of using of available starting materials, reaction products, which are either directly applicable as energy carriers or which are then, after further intermediate steps, applicable as energy carriers.

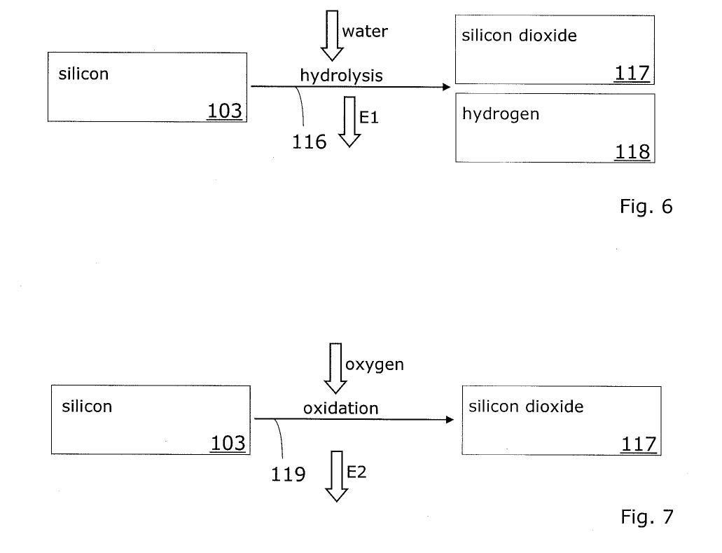

The term energy carrier is used herein to designate compounds, which can be used either directly as fuels or combustibles (such as, e.g., methanol 104 or hydrogen 118), and also for compounds (such as, e.g., silicon 103), which have an energy content or an elevated energy level and which can be converted in further steps with delivery of energy (refer to the energy E1 and E2 in the FIGS. 6 and 7) and/or with delivery of a further energy carrier (such as, e.g., hydrogen 118).

The transportability of the energy carrier is affected by the chemical reaction potential. For a safe transportability of the energy carrier, this reaction potential should preferably be low. In the case of silicon 103 as an energy carrier, specific framework conditions concerning the storage and transport should be obeyed in order to avoid initiating an undesired or uncontrolled reaction (oxidation) of the silicon. The silicon 103 should preferably be stored and transported in a dry state. In addition, the silicon 103 should not be heated because of the probability of a reaction with water vapor from the ambient air or with oxygen increases. Investigations have shown that silicon, up to approximately 300° C., has only a very low tendency to react with water or oxygen. It is ideal to store and transport the silicon 103 together with a water-getter (i.e. a compound that is hyrophillic/attracting water) and/or with an oxygen-getter (i.e. a compound attracting oxygen).

The term silicon-dioxide-containing starting material 101 is used herein to designate compounds which contain a large proportion of silicon dioxide (SiO₂). Sand and shale (SiO₂+[CO₃]²) are particularly suitable. Sand is a naturally occurring unconsolidated sedimentary rock and occurs everywhere on the surface of the Earth in more or less large concentrations. A majority of the occurrences of sand consist of quartz (silicon dioxide; SiO₂).

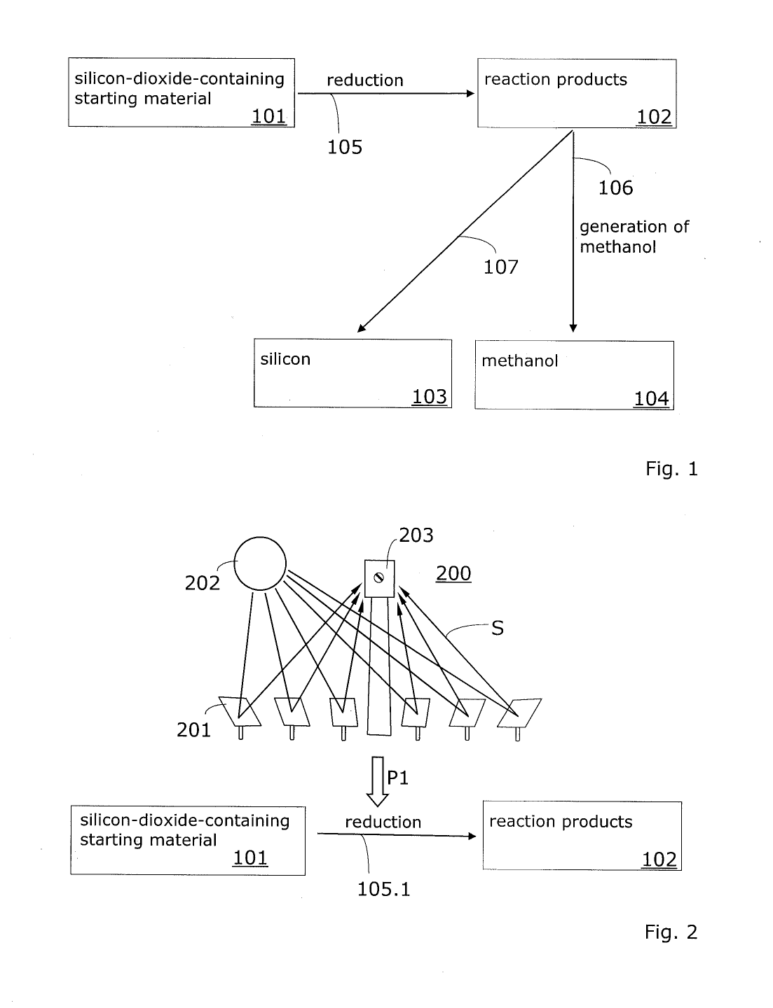

In FIG. 1, the basic steps of a first method according to the invention for providing storable and transportable energy carriers 103, 104 are shown. In this method, silicon 103, as a first storable and transportable energy carrier, and methanol 104, as a second storable and transportable energy carrier, are provided. The method comprises at least the following steps.

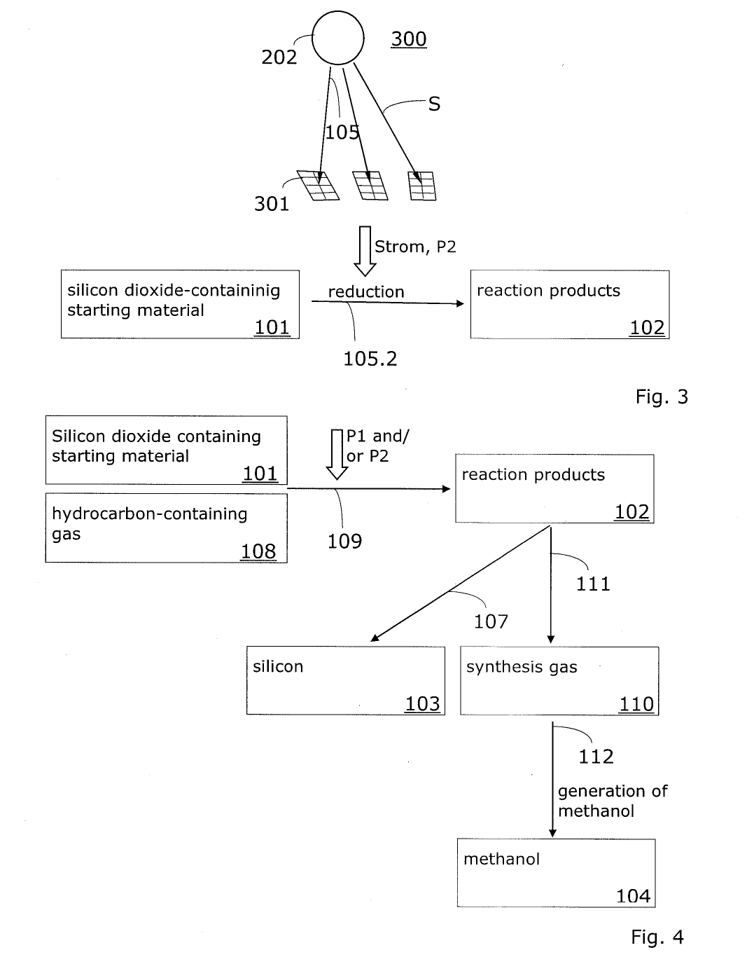

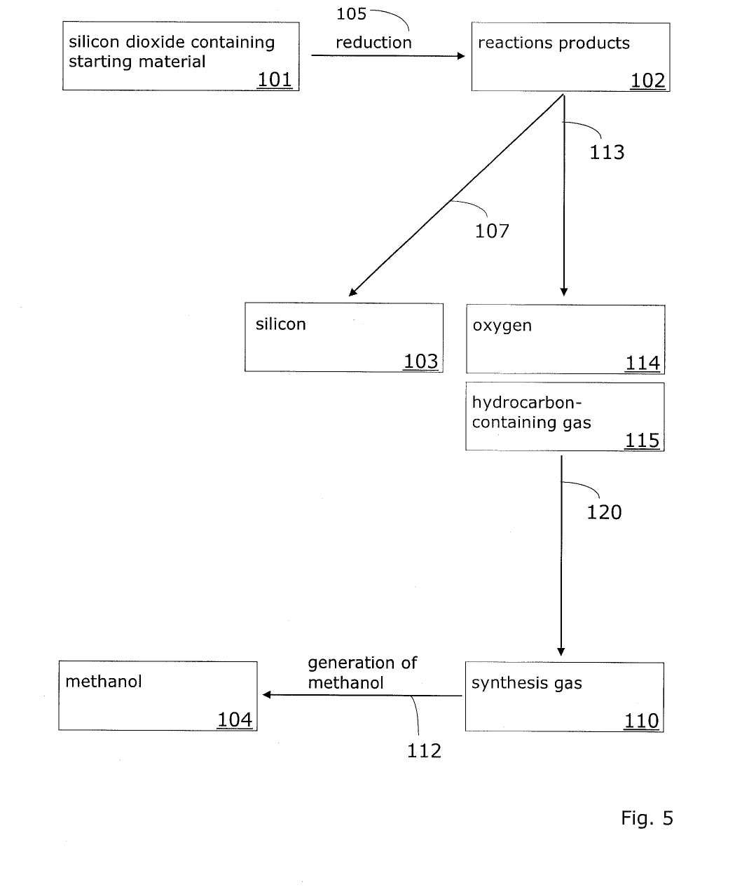

By a transformation, a silicon-dioxide-containing starting material 101 is converted to elementary silicon 103 by means of a reduction process 105. The elementary silicon 103 is called silicon for reasons of simplicity. According to the invention, the required primary energy (refer to primary energy P1 in FIG. 2 or primary energy P2 in FIG. 3) for this reduction process 105 is provided from a renewable energy source. In a subsequent step, at least a portion of the reaction products 102 of the reduction process 105 are utilized in a process 106 for generating methanol. In this process 106 for generating methanol, a synthesis gas 110 composed of carbon monoxide (CO) and hydrogen (H₂) comes to operation (see, FIGS. 4 and 5). In FIG. 1 it is further indicated schematically that the silicon 103 can be extracted from the process as the first energy carrier. The extraction of the silicon 103 is characterized in FIG. 1 as method step 107. The silicon 103 can, for example, be stored or transported away.

The transformation 105 is preferably a thermo-chemical transformation 105.1 (with participation of heat energy), as indicated schematically in FIG. 2, or an electro-chemical transformation 105.2 (with participation of electric current), as indicated schematically in FIG. 3.

In the thermo-chemical transformation 105.1 according to FIG. 2, the primary energy P1 for the transformation is delivered by sunlight S. For the thermo-chemical transformation 105.1, a solar thermal plant 200 is utilized, as indicated schematically in FIG. 2. The solar thermal plant 200 comprises a plurality of rotatable heliostats 201 which can preferably be tracked with the movement of the sun 202. The heliostats 201 reflect the sunlight S in the direction of a solar tower 203. In the focal point of the sunlight S, extremely high temperatures are achieved. In FIG. 2, it is indicated schematically by a block arrow P1 that the heat energy, which is provided by the solar thermal plant 200, comes to initiate and energize the endothermal reduction process 105.1. Depending on the embodiment, the solar energy can act directly on the silicon-dioxide-containing starting material 101, or a liquid transfer medium can be utilized as a facilitator for the dissemination/transfer of the energy P1.

In the electro-chemical transformation 105.2 according to FIG. 3, the primary energy P2 for the transformation is delivered by electric current, which is produced from sunlight S. For the electro-chemical transformation 105.2, a solar power plant 300 is applied, as indicated schematically in FIG. 3. The solar power plant 300 comprises a plurality of (rotatable) solar modules 301 which can preferably be tracked with the movement of the sun 202. The solar modules 301 convert the sunlight S to electric current. The electro-chemical transformation 105.2 can, for example, be performed by utilizing silicon dioxide as an electrode. A metal is utilized as a second electrode. As an electrolyte, for example, calcium chloride (CaCl₂) is utilized. This electro-chemical transformation process 105.2 works particularly well with a porous electrode made of silicon dioxide, which can, for example, be sintered from silicon dioxide. Details concerning this method can be taken from the following publications:
Nature materials 2003 June; 2 (6): 397-401, Nohira T., Yasuda K., Ito Y., Publisher: Nature Pub. Group;
“New silicon production method with no carbon reductant”, George Zheng Chen, D. J. Fray, T. W. Farthing, Tom W. (2000);
“Direct electrochemical reduction of titanium dioxide to titanium in molten calcium chloride”, George Zhen Chen, D. J. Fray, T. W. Farthing, Nature 407 (6802): 361-364; doi:10.1038/35030069;
“Effect of electrolysis a potential on reduction of solid silicon dioxide in molten CaCl₂”, YASUDA Kouji; NOHIRA Toshiyuki; ITO Yasuhiko; The Journal of physics and chemistry of solids, ISSN: 0022-3697, International IUPAC Conference on High Temperature Materials Chemistry No. 11, Tokyo, Japan (May 19, 2003), 2005, vol. 66, vo. 2-4 (491p.);
U.S. Pat. No. 6,540,901 B1;
WO 2006 092615 A1.

Preferably, the reduction process 105.1 is performed at a temperature of approximately 1900 degree Kelvin (=1630° C.) in order to reduce the silicon dioxide to silicon (Si). In the electro-chemical transformation 105.2, significantly lower temperatures (preferably less than 500° C.) are required.

Preferably, the reduction processes 105, 105.1, 105.2 are performed in an oxygen-poor or an oxygen-free environment, because otherwise the elementary silicon 103, which is produced in the reduction, would oxidize again immediately. In addition, the oxygen, together with the silicon, forms a layer of silicon dioxide on the melt, which could hinder the reduction process.

A further method according to the invention is shown in FIG. 4. Here, the reduction process 109 is carried out under supply of a hydrocarbon-containing gas 108. Preferably, methane (CH₄), biogas or natural gas (natural gas: NG) is utilized as the hydrocarbon-containing gas 108. In the reduction process 109, the following reaction products are generated:

silicon 103,

carbon monoxide and

hydrogen.

The term biogas is used herein to denominate gases which can be generated by fermentation processes under exclusion of air. Examples of biogas are the gases from sewage purification plants, from the keeping of useful animals, but also gases which can be provided from facilities which convert biomass. Here, preferably, only biogases are used which originate from renewable sources and which are not in concurrency with the cultivation of food products.

The methane mentioned should also originate preferably from renewable sources, which are not in concurrency with the cultivation of food products. The methane can, for example, be produced in a pyrolysis process, wherein the pyrolysis process is energized using biomass.

In this fourth method according to the invention, the hydrocarbon-containing gas 108 is utilized on one hand to serve as a reduction agent for the reduction of the silicon dioxide. On the other hand, the hydrocarbon-containing gas 108 serves as a “starting material” for the provision of the synthesis gas composed of carbon monoxide and hydrogen. The following reaction (1) takes place according to FIG. 4:

SiO₂+CH₄(g) [symbol] Si+2CO+4H₂(g) (1)

The reaction equation (1) reflects a method according to FIG. 4 in which methane is utilized as a hydrocarbon-containing gas 108. The “breakdown” of CH₄ in the synthesis gas 110 requires a supply of energy. Here, the corresponding energy [Δ_{R}H approx. 160 kJ/mol] is delivered from renewable energy sources. That is, the CH₄ is not utilized here as an energy supplier for this step 109. In order to be able to carry out this reaction, the energy must be supplied from the outside. In FIG. 4, the energy supply is indicated by a block arrow labeled with P1 and/or P2. That is, the energy can originate, e.g., from a solar thermal plant 200 and/or from a solar power plant 300.

In the method according to FIG. 4, the silicon dioxide of the silicon-dioxide-containing material 101 functions as the donor of oxygen.

Here, the synthesis gas 110 (2CO+4 H₂(g)) is further converted to methanol 104 in a process 112 for the generation of methanol.

A further method according to the invention is shown in FIG. 5. A scheme is illustrated which corresponds in part to the method of FIG. 1. However, further method steps are appended here with respect to the method of FIG. 1. Here, in the reduction process 105, silicon 103 and oxygen 114 are generated as the reaction products 102. Here, the oxygen 114 is converted using a supply of a hydrocarbon-containing gas 115 to a synthesis gas 110 composed of carbon monoxide and hydrogen. The method step 120 concerns a gas oxidation process. The gas oxidation process is slightly exothermal. Preferably, methane (CH₄), biogas or natural gas (NG) is utilized as the hydrocarbon-containing gas 115. Here, the synthesis gas 110 is then also converted to methanol 104 in a process 112 for generating methanol.

In connection with the FIGS. 6 and 7, silicon 103 can be utilized as an energy carrier. The reduced silicon 103 is an energy-rich compound. This silicon has the tendency to oxidize with water in liquid or vapor form again to silicon dioxide 117, as shown schematically in FIG. 6. In the so-called hydrolysis 116 of the silicon 103, energy E1 is liberated, because an exothermal reaction is concerned. In addition to the silicon dioxide 117, hydrogen is generated, which can, for example, be utilized as an energy carrier or fuel. Preferably, the hydrolysis 116 takes place at elevated temperatures. Temperatures are preferred which are significantly above 100° C. In a temperature range between 100° C. and 300° C., a conversion in usable quantities is achieved in cases when the silicon, in a very fine-grained or a powdery consistency, is brought in contact with water vapor and is stirred. Since otherwise silicon up to above 300° C. has only a very low tendency to react with water, the hydrolysis 116 is preferably performed at temperatures in the temperature range between 300° C. and 600° C.

According to the invention, in a method according to FIG. 6, the silicon is introduced into a reaction area and is mixed with water in liquid or vapor form. In addition care is taken according to the invention that the silicon 103 has a minimum temperature. The silicon 103 is either heated (e.g. using heating means or by means of heat-generating or heat-delivering additives) or the silicon 103 is already at a corresponding temperature level when it is introduced.

Under these framework conditions, hydrogen is then liberated in the reaction area as a gas. The hydrogen is extracted from the reaction area.

In the following, a numerical example for a method according to FIG. 1 in combination with FIG. 6 or according to FIG. 5 in combination with FIG. 6 is given:

1 mol (=60.1 g) SiO₂ forms 1 mol (=28 g) Si. 1 mol (=28 g) Si in turn forms 1 mol (=451 g) H₂. That is, 2.15 kg SiO₂ form 1 kg Si, and from this 1 kg Si, 1.6 m³ H₂ are formed.

The silicon 103, however. Also has the tendency to oxidize again with oxygen to silicon dioxide 117, as represented in FIG. 7. An energy E2 is liberated, because an exothermal reaction is concerned. Preferably, the oxidation 119 takes place in a temperature range between 500° C. and 1200° C. Temperatures are preferred which are above 1000° C. The corresponding temperature can be provided by means of a solar thermal plant 200 or a solar power plant 300.

The method according to FIG. 7 can be performed, for example, in an oxidation oven. Preferably, in the oxidation oven, a thermal oxidation is performed in which the energy for initiating/energizing the oxidation originates from renewable energy sources (preferably from solar energy).

The oxidation of the silicon 103 should preferably take place using dry oxygen in order to exclude a simultaneous concurrent hydrolysis process.

The method according to FIG. 7 can, for example, also be performed in a plasma oxidation oven. Here, only temperatures in the temperature range between 300° C. and 600° C. are necessary, because a portion of the required energy is provided by the plasma.

The generation of methanol can be performed according to one of the methods which are known and utilized at large scale. A method is preferred in which a catalyst (e.g. a CuO—ZnO—Cr₂O₃ or a Cu—Zn—Al₂O₃ catalyst) is applied.

The invention has the advantage that in the reduction of the silicon dioxide, no CO₂ is liberated. The required energy is provided from renewable energy sources, preferably from solar energy plants 200 or 300.

The elementary silicon 103 is applied preferably in powder form or in granular or grainy form, so as to offer a preferably large surface in the oxidation (refer to step 119 in FIG. 7) or in the hydrolysis (refer to step 116 in FIG. 6).

Silicon plays an essential role for electronic components, such as solar cells and semiconductor chips, as well as for the production of polysiloxanes. The elementary silicon 103 can thus also be processed for other purposes.

1. A method for providing storable and transportable energy carriers the method comprising the following steps:
transformation of a silicon-dioxide-containing starting material to silicon in a reduction process, wherein the primary energy for this reduction process is provided from a renewable energy source,
applying a portion of the reaction products from the reduction process in a process for the generation of methanol, wherein in the process for the generation of methanol, a synthesis gas composed of carbon monoxide and hydrogen is used.

2. The method according to claim 1, wherein the transformation is a thermo-chemical or an electro-chemical transformation.

3. The method according to claim 2, wherein the primary energy for the transformation is provided by sunlight from a thermo-chemical transformation in a solar heat plant or an electro-chemical transformation in a solar power plant.

4. The method according to claim 1, wherein the reduction process is carried out at a temperature of approximately 1.900° Kelvin (=1.630° C.).

5. The method according to claim 1, wherein the reduction process is carried out in an oxygen-poor or an oxygen-free environment.

6. The method according to claim 1, wherein the reduction process is carried out under supply of a hydrocarbon-containing gas selected from the group consisting of methane, biogas and a natural gas and the following reaction products from of the reduction process are used:
silicon
carbon monoxide and
hydrogen.

7. The method according to claim 6, wherein the silicon is provided as a first storable and transportable energy carrier and methanol is provided as a second storable and transportable energy carrier.

8. The method according to claim 6, wherein energy for converting the hydrocarbon-containing gases are provided from solar energy.

9. The method according to claim 1, wherein the following reaction products of the reduction process are provided:
silicon and
oxygen.

10. The method according to claim 9, wherein the silicon is provided as a first storable and transportable energy carrier.

11. The method according to claim 9, wherein methanol is produced from carbon monoxide and hydrogen is used as a second storable and transportable energy carrier.

12. The method according to claim 1, wherein the silicon is provided as a first storable and transportable energy carrier and wherein, in a further step water or water vapor is brought in contact with the silicon so as to provide hydrogen, silicon dioxide and a first amount of energy in a hydrolysis reaction.

13. The method according to claim 1, wherein the silicon is provided as a first storable and transportable energy carrier and wherein in a further step oxygen is brought in contact with the silicon so as to provide silicon dioxide and a second amount of energy in an oxidation reaction.
